(19) United States
(12) Patent Application Publication
(10) Pub. No.: US 2006/0089599 A1
(43) Pub. Date: Apr. 27, 2006

(54) Blood donor needle assembly and cover

(76) Inventors: Daniel Lynn, Spring Grove, IL (US); Larry Servi, Hawthorn Woods, IL (US); Scott Ariagno, Mundelein, IL (US); Dale Ellis, Ingleside, IL (US); Michael T. K. Ling, Vernon Hills, IL (US); Daniel F. Bischof, Bull Valley, IL (US); Suchuam C. Fan, Libertyville, IL (US); Adel Sadik, Fox River Grove, IL (US)

Correspondence Address: BAXTER HEALTHCARE CORPORATION, ONE BAXTER PARKWAY, DF2-2E, DEERFIELD, IL, 60015, US

(21) Appl. No.: 10/974,651

(22) Filed: Oct. 27, 2004

(51) Int. Cl. A61M 5/00

(52) U.S. Cl. 604/111

(57) ABSTRACT

Medical needle assemblies and needle covers for medical needles are disclosed. The needle covers include a relatively rigid outer surface and more resilient inner surface. The needle covers protect the needle from damage, allow for effective sterilization of the assembly, provide a sterile barrier, and evidence of product tampering.

The present invention relates to a medical needle assembly of the type commonly used in both manual and automated blood donations. More particularly, the present invention relates to a needle cover for a medical needle that provides evidence of product tampering, allows for sterilization of the needle, and protects the needle from damage, such as bending.

Blood collection kits, in their most basic form, typically include a collection container for receiving blood from a donor, a tube that provides a flow path from the blood donor to the collection container and a hypodermic needle attached to one end of and in flow communication with the tubing. The needle assembly typically includes a needle attached to a needle hub which allows for manipulation of the needle assembly by the technician.

Donor needles are commonly shielded before and after their use with a removable needle cover. Shielding the needle protects the technician from inadvertent contact with the sharpened needle tip. Shielding the needle also protects the needle from damage during shipping and transport. Shielding also preserves the sterility of the needle prior to use and ideally maintains the integrity of the closed system by preferably providing a hermetic seal between the needle cover and needle assembly. The needle cover may also provide assurance to the end user that the needle has not been tampered with.

Needle covers typically include an elongated sleeve with an inner bore for receiving the needle. The needle cover is typically made of a plastic material with sufficient rigidity to protect the needle from damage, such as, but not limited to, bending. Preferably, the plastic material is permeable to moist heat, allowing for steam sterilization, the preferred form of sterilizing many medical products. The needle cover seals to the needle hub post in a way that provides the user with evidence of tampering.

Examples of needle assemblies including associated needle covers are provided in U.S. Pat. No. 4,402,682 and U.S. Pat. No. 4,496,352, both of which are assigned to the assignee of the present application and incorporated herein by reference. These patents disclose a needle assembly including a cannula, (i.e., needle) attached to a hub and to donor tubing. The needle covers are made of a plastic material, which forms a thermal bond with the post of the needle hub to provide a tamper evident seal. The needle covers described in the above-referenced patents also include an internal plug within the bore of the needle cover and located at the distal end of the needle cover. The plug is made of a resilient material and includes a pocket for enveloping and protecting the distal needle tip.

Another example of a needle assembly and associated needle cover is disclosed in U.S. Pat. No. 4,551,138. The needle cover disclosed therein includes a hollow body made of a sterilizable plastic material. The needle cover includes a resilient “layer” made of a polymeric elastomer located near the proximal open end of the needle cover. The diameter of the cylindrical resilient layer at the proximal end of the cover has a diameter that is smaller than the diameter of the needle post engaged by the needle cover. The needle cover is placed over the needle and engages the post of the needle hub. According to U.S. Pat. No. 4,551,138, a hermetic seal is formed between the needle cover and the hub or post portion of the needle assembly.

While the above-described needle covers have worked satisfactorily, they are not without drawbacks. For example, the need for a needle cover that is sufficiently rigid to protect the needle from damage (which may occur during shipping) often means having to use a plastic material that is less than ideally suited for effective steam sterilization. Consequently, this may result in longer sterilization cycles which may have a deleterious effect on other components of the blood processing set. In addition, a needle cover made of a more rigid plastic may also be less desirable in that contact between the needle tip and the hard plastic can result in dulling of the needle tip. On the other hand, while a needle cover made of different, preferably more resilient plastic may be more desirable from a sterilization and tip protection standpoint, it may not offer sufficient stability and protection against bending or bowing.

Thus, it would be desirable to provide a needle cover that is sufficiently rigid to protect the needle from damage or bending, but that still allows for effective sterilization, can still provide evidence of tampering, and is cost effective to manufacture.

### SUMMARY OF THE INVENTION

In one aspect, the present invention is directed to a needle assembly that includes a hub assembly, a needle having a distal end and a proximal end, attached to the hub assembly at its proximal end. The needle assembly further includes an attachable and removable needle cover for enclosing the needle. The cover includes an elongated inner sleeve portion and elongated outer sleeve portion whereby the inner sleeve portion engages the hub assembly and provides evidence of tampering.

In another aspect, the present invention is directed to a needle cover for a medical needle. The needle cover includes a proximal end and a distal end. The needle cover further includes an outer sleeve portion made of a first plastic material having an open proximal end, and an inner sleeve portion associated with the outer sleeve portion that likewise includes an open proximal end. The outer sleeve portion has an inner diameter greater than the outer diameter of the inner sleeve portion at their proximal ends.

In another aspect, the present invention is directed to a needle cover for a medical needle where the cover includes a proximal end and a distal end. The needle cover includes an outer sleeve portion made of a first material having an open proximal end and an inner sleeve portion made of a second plastic material having an open proximal end. The needle cover further includes at least one window in the outer sleeve portion through which the inner sleeve is exposed.

In a further aspect, the present invention is directed to a needle cover for a medical needle that includes an elongated body having an outer surface and an inner surface and an open proximal end for receiving a needle and a closed distal end. The body of the needle cover defines an interior bore. The inner surface has a modulus lower than the modulus of the outer surface.

In another aspect, the present invention is directed to a medical needle assembly including a needle cover and a needle hub assembly. The needle hub assembly includes a hub and a needle post that extends through the interior of the hub. A medical needle is attached to the post. The post includes a beveled sealing ring adapted for engaging the needle cover. The hub includes gripping members on the outer surface thereof.

### BRIEF DESCRIPTION OF THE DRAWINGS

FIG. 1 is a plan view of a disposable blood collection kit.

FIG. 2 is a perspective view of the needle assembly with the cover attached, embodying the present invention.

FIG. 3 is a cross-sectional side view of the needle assembly of FIG. 2.

FIG. 4 is an end view of the needle cover of FIG. 2.

FIG. 5 is a perspective view of the needle assembly embodying the present invention with the cover removed.

FIG. 6 is a side view of the needle assembly of FIG. 5.

FIG. 7 is an end view of the needle assembly of FIG. 5.

FIG. 8 is a partial side view of the needle hub assembly and a pre-attached hollow needle.

FIG. 9 is a cross-sectional side view of a needle cover embodying the present invention.

FIG. 10 is an enlarged cross-sectional side view of the proximal end of the needle cover of FIG. 9.

FIG. 11 is a partial cross-sectional side view of the needle cover embodying the present invention engaging the post of the needle hub.

FIG. 12 is an exploded perspective view of a two-part needle cover embodying the present invention.

FIG. 13 is a proximal end view of the needle cover of FIG. 9.

FIG. 14 is a perspective view of the needle assembly with an alternative embodiment of the needle hub.

FIG. 15 is a perspective view of the needle assembly with another alternative embodiment of the needle hub.

FIG. 16 is a perspective view of a needle assembly with still another alternative embodiment of the needle hub.

FIG. 17 is a distal end view of the needle assembly of FIG. 16; and

FIG. 18 is a perspective view of the needle assembly with still another alternative embodiment of the needle hub.

FIG. 19 is a partial view of the needle assembly and needle cover (in cross-section) prior to initial capping and steam sterilization.

FIG. 20 is a partial view of the needle assembly and needle cover (in cross-section) after initial capping.

FIG. 21 is a partial view of the needle assembly and needle cover (in cross-section) after sterilization and uncapping.

### DETAILED DESCRIPTION OF THE DRAWINGS

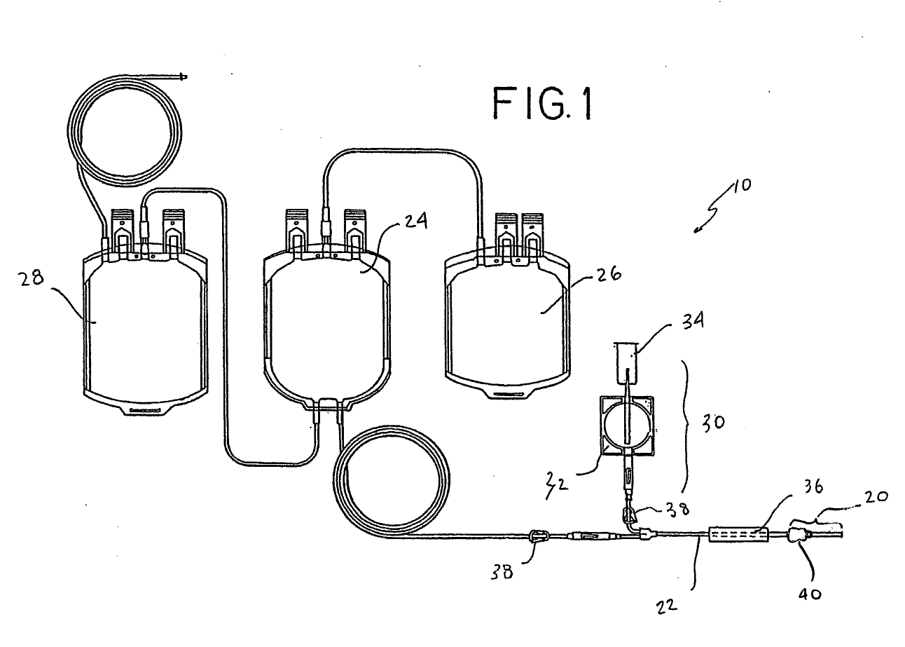

Turning now to the Figures, FIG. 1 shows a disposable blood collection and processing kit of the type commonly used in whole blood donations. Disposable set 10 includes a needle assembly 20 attached to a plastic tube 22, which provides a flow path to collection container 24. Collection kit 10 may further include satellite containers 26 and 28 for receiving separated blood components or for holding a storage medium for a separated component, as will be recognized by those of skill in the art. The system shown in FIG. 1 is just one example of a blood collection processing set. Other examples are shown and described in, for example, U.S. patent application Ser. No. 10/956,296, filed Oct. 1, 2004 and incorporated herein by reference in its entirety.

Set 10 may further include a sampling unit, generally designated by reference numeral 30, for collecting samples of the donated blood. Sampling unit 30 may include a sampling container 32 and a sample tube holder 34. Flow control through the disposable set 10 is achieved by manipulating flow control clamps 38. Details of the blood collection, the collection set and blood sampling are described in U.S. Pat. No. 6,387,086, which is incorporated herein in its entirety by reference, and in the aforementioned U.S. patent application Ser. No. 10/956,296, previously incorporated by reference.

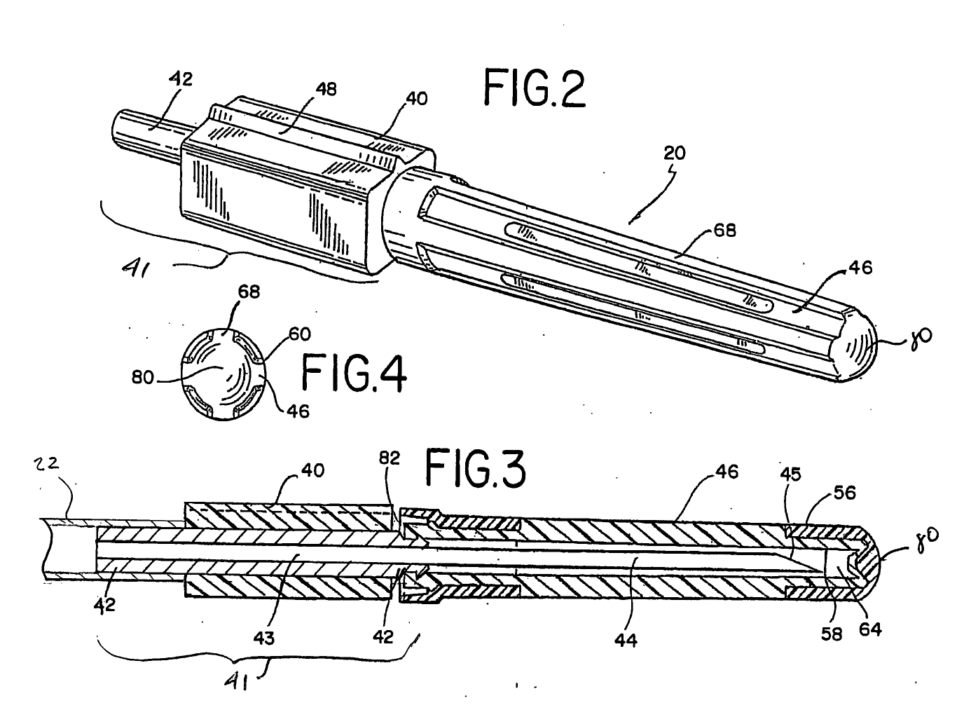

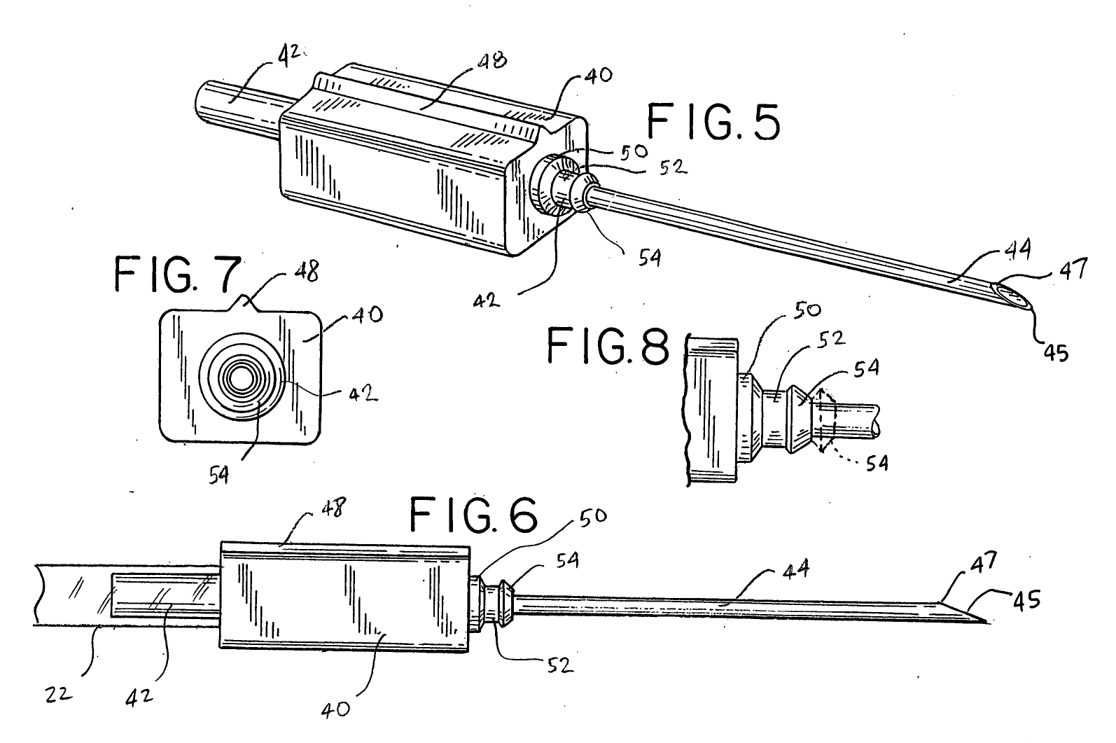

FIG. 2 shows a needle assembly 20 embodying the present invention. Assembly 20 includes a cannula or needle 44 (shown in FIG. 3) adapted for insertion into the vein of the blood donor. Needle 44 is typically made of stainless steel or other alloy metal. Needle 44 may be further coated by one or more lubricants to facilitate venipuncture. Needle lubricants include, but are not limited to cross-linked, silicone based lubricants and/or simple lubricating oils that will be known to those of skill in the art. As seen in FIG. 5, needle 44 is hollow and terminates at its distal end in a sharpened and beveled end 45.

Needle 44 is attached to hub assembly 41. Hub assembly 41 includes hub 40, which is compact and easily manipulated between the fingertips of the technician. Hub assembly 41 may further include needle post 42 which, as shown in FIG. 3, is essentially a tubular member that includes a bore for receiving the shank of needle 44, described in greater detail below.

Hub 40 further includes an inner bore 43, for receiving hollow needle post 42 which receives or is otherwise attached to needle 44. As shown in FIG. 3, needle post 42 extends through the body of hub 40 within which it is typically press-fit. The distal end of post 42 that extends beyond hub 40 is joined to tube 22, thus providing a continuous flow path from the needle tip to tube 22 (and, ultimately, to collection container 24).

In one embodiment, hub 40 may include an outwardly projecting ridge 48, which extends axially along one exterior surface of hub 40. Ridge 48 serves as a reference point for the proper alignment of the beveled end 45 of needle 44 during assembly, as shown, for example, in FIG. 5. In addition to facilitating the initial alignment of the beveled end 45 of the needle 44 during assembly, ridge 48 also functions as a visual guide for the phlebotomist during venipuncture and the phlebotomy. Proper bevel orientation is desirable, because it ensures the correct orientation of the needle for venipuncture. Orienting needle 44 with needle heel 47 facing up also improves the blood flow through needle 44 (see FIG. 5).

Hub 40 is preferably made of a flexible material, compressible by the fingers of the technician. This enables the technician to effect a firm finger grip on the hub 40 and to carefully maneuver and control the hub for fast and comfortable venipuncture. Preferred materials for hub 40 include polyvinylchloride or thermoplastic elastomers. Particularly preferred is low modulus and low durometer polyvinylchloride.

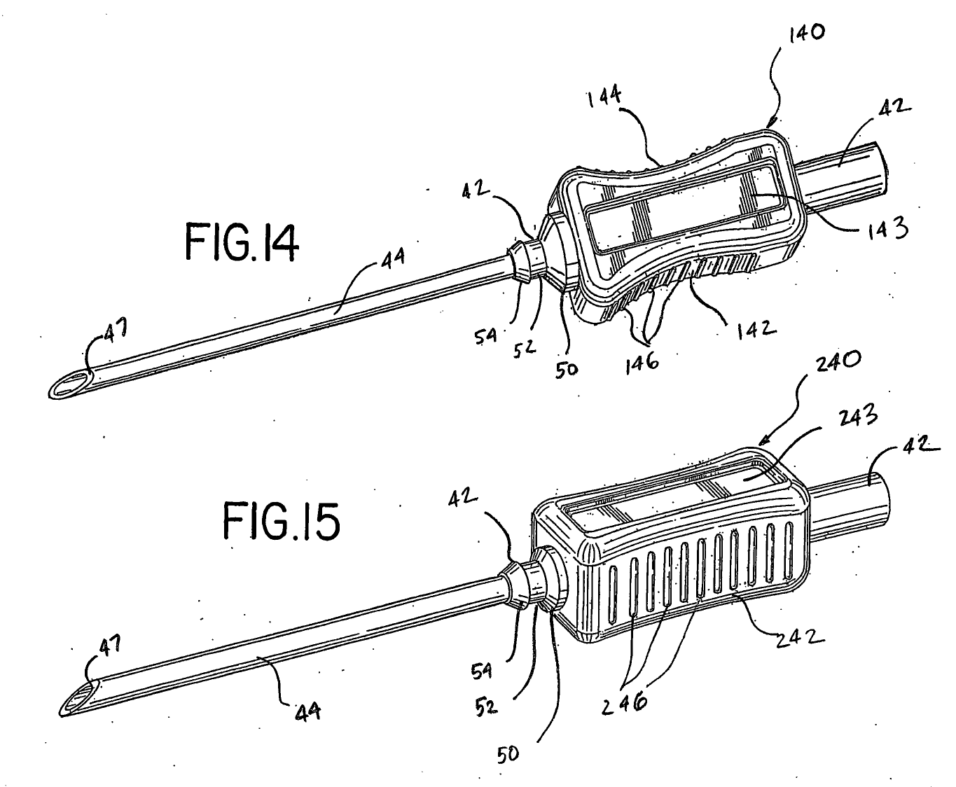

Alternative embodiments of hub 40 are shown in FIGS. 14-18, which include additional ergonomically desirable features. For example, hub 140 shown in FIG. 14 includes concave sidewalls 142 and 144 which allow for comfortable gripping with the thumb and forefinger. Sidewalls 142 and 144 may further include gripping members on the surfaces thereof. Gripping members may be molded or embossed projectors on the surfaces of hub 140 that affect the contour of the hub surface. Improved gripping surfaces can also be provided by treating the surfaces of hub 140, such as by making it coarser or less smooth. In one embodiment, gripping members may be spaced apart raised ribs 146 on the surfaces 142 and 144 of hub 140.

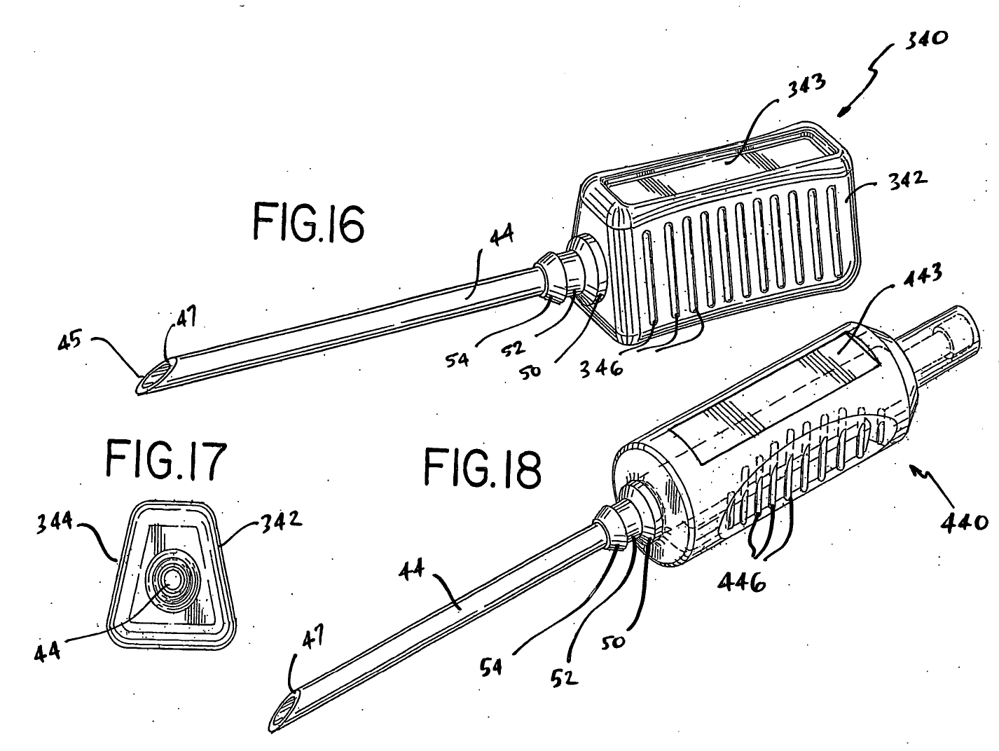

FIGS. 15 and 16 show alternative embodiments of hubs 240 and 340 respectively. In the hubs 240 and 340 of FIGS. 15-17, sidewalls 242, 244 (not shown) and 342, 344 are less concave and may even be substantially flat. Hubs 240 and 340 also may include gripping members or gripping means, such as ribs 246 and 346 on the surfaces of the sidewalls, as described above. Hub 346 may have a generally trapezoidal end profile, as shown in FIG. 17, again to provide for more comfortable gripping by the user.

FIG. 18 shows a generally cylindrical hub 440 which may also include concave sidewalls 442 and 444. As in the previous embodiments, sidewalls 442 and 446 may also include ribs 446 or other gripping members or means for improved gripping.

Proper needle orientation during assembly and venipuncture may be ensured by aligning the beveled end 45 of needle 44 with a selected surface of hubs 140, 240, 340 or 440. In one embodiment, hub surface 143, 243, 343 or 443 serves as a visual reference point to ensure that the needle bevel 45 and heel 47 are properly oriented (i.e., facing up). Although not shown in FIGS. 14-18, surfaces 143, 243, 343 and 443 may be molded, embossed, or printed with the name of the needle, manufacturer or other indicia.

Turning briefly to FIGS. 5 and 6, needle post 42 projects outwardly from the distal end of hub 40 and is attached to the proximal end of needle 44. In one embodiment, post 42 may be overmolded over the proximal end of needle 44. In another embodiment, the proximal end of needle 44 may be inserted into the hollow bore of post 42 and secured to post 42 by adhesive bonding. A preferred adhesive may be one that is curable by ultraviolet (UV) radiation. Other ways of combining or otherwise attaching needle 44 and post 42 that will be recognized by those of skill in the art may also be used.

As shown in FIGS. 5 and 6, proximal end of needle post 42 includes a base portion 50, an intermediate portion 52 of reduced diameter relative to the base portion 50, and a sealing ring 54 at the distal-most end of post 42. Sealing ring has a diameter greater than intermediate portion 52, and is beveled, as shown. In an alternative embodiment, needle post 42 may include a multiple sealing ring arrangement (i.e., two or more sealing rings), as shown in broken lines in FIG. 8. As described below, sealing rings 54 provide a surface to which the needle cover may be bonded.

The needle assembly of the present invention further includes an attachable and removable cover 46. As shown in FIGS. 2 and 3, cover 46 is preferably in the form of an elongated tube including a closed distal end 80 and an open proximal end 82. Cover 46 may have a slightly conical shape, as shown in the Figures.

Outer surface 56 of cover 46 may include one or more gripping members or gripping means to provide the user with a better grip of needle cover 46 during capping, recapping and uncapping. In one embodiment, outer surface 56 of cover 46 may be made with or subsequently treated to provide a rougher or coarser surface. In another embodiment, outer surface 56 may be provided with one or more gripping members. In a preferred embodiment, gripping member(s) may be one or a plurality of axially extending raised ribs 68 which project from the outer surface 56 of cover 46. Ribs 68 facilitate gripping of cover 46 by the user during removal and capping. The number of ribs can vary, although in a preferred embodiment, four ribs equally spaced (i.e., separated by 90° intervals) are preferred. Cover 46 may also include one or more windows 66 between ribs 68, discussed further below.

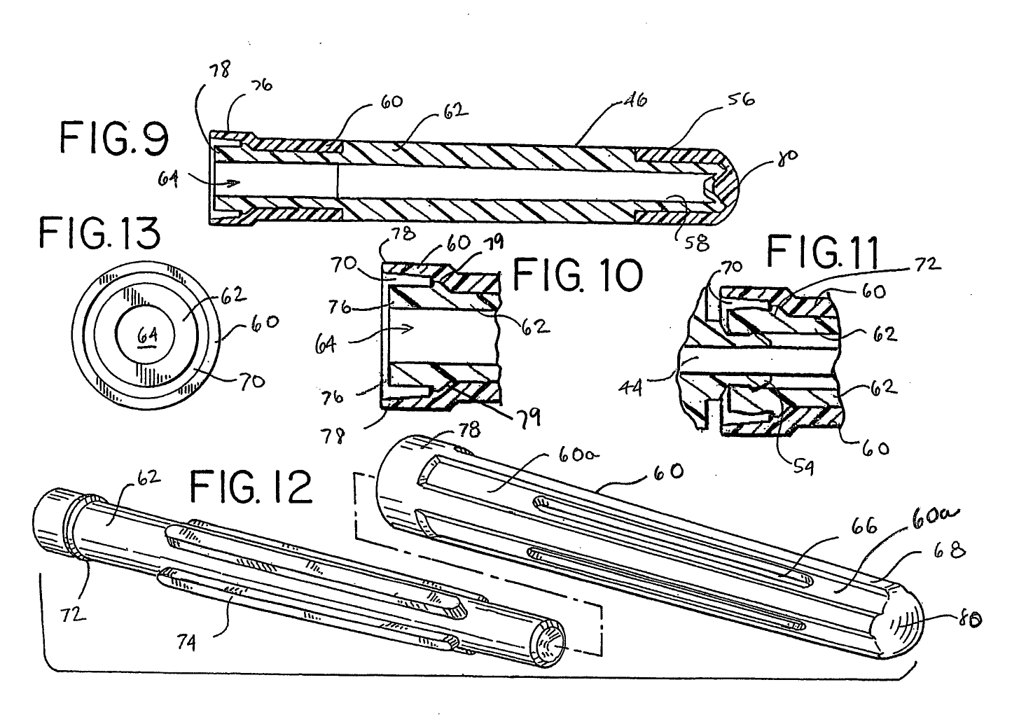

Turning now to FIGS. 9 and 12, cover 46 may be of one-piece or multiple-piece construction. As shown in FIGS. 9 and 12, cover 46 is preferably of two-piece integral construction. Cover 46 preferably includes an outer sleeve portion 60 and inner sleeve portion 62 within outer sleeve portion 60. Inner sleeve portion 62 includes internal bore 64 for receiving needle 44. In a preferred embodiment, cover 46 is made in a two-shot molding process. For example, in one embodiment, inner sleeve portion 62 is molded in a first molding operation. Molded inner sleeve portion 62 is then placed in a molding cavity and outer sleeve portion 60 is overmolded onto inner sleeve portion 62. Alternatively, outer sleeve 62 may be molded in a first “shot,” with inner sleeve formed by injecting the plastic material of inner sleeve portion 64 into the inner bore of sleeve 62 in a second “shot.” In another alternative method of manufacturing and assembling cover 46, both outer sleeve portion 60 and inner sleeve portion 62 may be separately molded and assembled together by mechanical attachment (with or without adhesive bonding). In one such embodiment, raised projections 74 (discussed below) or inner sleeve 62 may be snap fit into windows 66 (also discussed below) of outer sleeve portion 60.

In one preferred embodiment, outer sleeve portion 60 is made of a polymeric material such as, for example, polyolefin, acrylic or other plastic material that is sufficiently rigid to protect needle 44 from damage, and that is compatible with the material of inner sleeve portion 62. For example, in one embodiment, the plastic material of outer sleeve portion 60 may have a Young's modulus of at least approximately 50,000 psi and a preferred Young's modulus of approximately 50,000-450,000. More preferably, the plastic material of outer sleeve 60 may have a Young's modulus of approximately 80,000-220,000 psi. Preferred materials include, but are not limited to high density polyethylene, polyacetal, PET, polycarbonate, rigid and semi-rigid polyvinyl chloride and polypropylene.

The material of outer sleeve portion 60 may have a hardness of at least approximately 95 Shore A. In one embodiment, the hardness of the outer sleeve material may be between approximately 100 Shore A and up to, for example, a Rockwell Hardness of 120 R Scale. In a preferred embodiment, outer shell sleeve 60 may be made of polypropylene with a Rockwell Hardness (durometer) of approximately 110 R Scale.

In addition to providing the desired rigidity, the plastic material of outer sleeve portion 60 should also be one that will not stick to other parts of the disposable blood processing set as a result of the sterilization process or simply from storage of the product prior to use. In this regard, polyolefins such as polypropylene, are preferred.

Inner sleeve portion 62 may be made of a medical grade plastic material that preferably is softer (and, thus, has a lower modulus) than the material of outer sleeve 60, and is also sterilizable by moist heat sterilization (i.e., autoclaving). Preferably, the plastic material of inner sleeve 62 may have a Young's modulus of less than approximately 50,000 psi, with a preferred Young's modulus range of approximately 1,000-50,000 psi and a more preferred range of approximatley 5,000-35,000 psi. The plastic material of inner sleeve portion 62 may also have a durometer of approximately 60-90 Shore A, with a durometer of 70-85 Shore A being preferred. Examples of suitable materials include, but are not limited to, elastomers such as Hytrel® and more preferably, polyvinyl chloride.

Of course, it will be appreciated that the present invention is not limited to the plastic materials (with the hardness and tensile properties) described above. The needle cover of the present invention may include an outer surface or outer sleeve made of any relatively more rigid (i.e., relative to the material of inner surface or inner sleeve portion 62) plastic material sufficient to protect the needle from bending, and an inner surface or inner sleeve in proximity to the needle made of relatively more resilient (lower modulus) material. The materials selected should be capable of being sterilized by the selected form of sterilization (e.g., moist heat), and compatible with each other and with other parts of the needle assembly and blood processing set.

As shown in FIGS. 3 and 9, inner and outer sleeve portions 60 and 62 are preferably coterminous with each other. Inner sleeve 62 extends along the entire axial length of outer sleeve 60. As further shown in FIGS. 9 and 12, inner sleeve 62 may include at least one, and preferably a plurality of upstanding projections 74 on the outer surface thereof. Projections 74 are disposed within windows of outer sleeve 60, thereby exposing inner sleeve 62 to the outside environment through windows 66 of outer sleeve portion 60. As shown in FIG. 3, projections 74 are preferably flush with the outer surface of sleeve portion 60 in the recessed areas 60a (between ribs 68) in proximity to windows 66. This allows for more complete sterilization of needle assembly, including the needle housed therein, by exposing the inner sleeve portion 62 and interior regions of the needle assembly 20 to permeation by the moist heat of steam sterilization.

Turning now to FIGS. 10, 11 and 13, outer sleeve portion of needle cover 46 may be outwardly flared at proximal end 78 thereof. In addition, the inner surface of outer sleeve portion 60 also includes an outwardly extending step 79, as generally shown in FIG. 10. Outwardly extending step 79 defines a gap 70 between the internal surface of outer sleeve 60 and the outer surface of internal sleeve portion 62.

The internal diameter of inner sleeve 62 is preferably less than the outer diameter of sealing ring 54. However, because the inner sleeve 62 is typically made of a more resilient plastic material, such as polyvinylchloride, when needle 44 is inserted into cover 46, resilient inner sleeve 62 flexes outwardly as shown in FIG. 11. Thus, a snug interference fit between the inner sleeve 62 and post 42 is achieved. Gap 70 provides space for the expansion of proximal end of inner sleeve 62 during placement of cover 44 onto post 42. In addition, gap 70 and the clearance provided thereby reduces the required removal torque.

Because the inner sleeve material (example, polyvinylchloride) is generally compatible with the polymeric material of post 42 (e.g., polycarbonate), during steam sterilization, a thermal bond is formed along the concentric interface of the inner surface of inner sleeve 62 and sealing ring 54. The beveled surface of ring(s) 54 further serve(s) as energy directors during bonding. The thermal bond created is sufficient to allow for removal of cover 46 at the time of use without requiring excessive removal torque, while still providing a sufficient amount of resistance to indicate whether the needle assembly has been tampered with. In addition, the bond formed between post 42 and inner sleeve 62 serves as an effective sterile barrier in that it prevents ingress of bacteria and preserves the sterility and integrity of the closed system. In a preferred embodiment, cover removal should require between approximately 2-24 in.-oz. torque to break the bond.

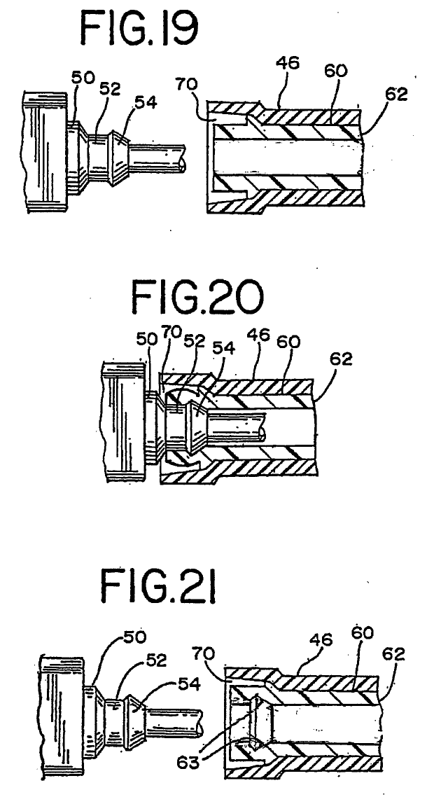

FIGS. 19-21 show the shape of inner sleeve 62 before initial capping (FIG. 19), during capping (FIG. 20) and after sterilization and uncapping (FIG. 21). As shown in FIGS. 19-21, after, and as a result of steam sterilization, the inner surface of inner sleeve portion 62, in the area of sealing ring 54, may generally retain the shape in which it conforms to sealing ring 54. Stated differently, exposure of the resilient material of inner sleeve portion 62 to the heat of sterilization results in the formation of seat 63 (shown in FIG. 21) that can engage sealing ring 54 and thereby allow cover 46 to lockingly engage post 42 (to prevent cover 46 from slipping off needle assembly 41) during subsequent capping. And audible “click” confirms for the user that cover 46 is lockingly engaged and securely reattached to post 42.

The present invention provides several benefits over known and presently used needle covers. For example, the present invention provides a needle cover with a substantially rigid outer shell or surface that protects the needle from bending or other possible damage. At the same time, needle cover 46 provides a softer, more resilient interior surface along the entire axial length of the needle 44 which prevents damage to the needle shaft and dulling of the needle tip (which would be possible, due to contact with an otherwise rigid plastic material). The needle cover of the present invention provides these advantages without diminishing the effectiveness of steam sterilization or requiring longer sterilization cycles by allowing for substantial permeation of the needle assembly by the moist heat of the sterilization process (e.g., through windows 66). Thus, the needle can be protected from damage by a relatively rigid outer surface and the needle assembly can still be sufficiently and adequately exposed to the moist heat of steam sterilization, thereby ensuring complete and thorough sterilization of the needle assembly.

In addition, the two-piece construction of the needle cover of the present invention and the preferred molding process by which it is formed (including the proximal gap between the outer and inner sleeves) allows for the secure and hermetic sealing of the cover to the needle post in a way that does not unduly stress the needle cover and results in acceptable removal torque.

The needle assembly of the present invention has been described in the context of its preferred embodiments. The description set forth above is by no means intended to limit the invention, which is recited in the appended claims. Further advantages of the present invention will be apparent to those of ordinary skill in the art.

What is claimed:

1. A medical needle assembly comprising:
a needle hub assembly;
a needle having a distal end and a proximal end attached to said hub assembly;
an attachable and removable needle cover for enclosing said needle cover comprising:
an elongated inner sleeve portion; and
an elongated outer sleeve portion;
whereby said inner sleeve portion engages said hub assembly and provides evidence of tampering.

2. The needle assembly of claim 1 wherein said inner and outer sleeves are generally cylindrical.

3. The needle assembly of claim 1 wherein said inner sleeve portion includes a bore for receiving said needle.

4. The needle assembly of claim 3 wherein the proximal end of said inner sleeve is coterminous with the proximal end of said outer sleeve.

5. The needle assembly of claim 1 wherein said outer sleeve portion is generally conically shaped.

6. The needle assembly of claim 5 wherein the inner surface of said outer sleeve includes an outwardly extending step at the proximal end of said outer sleeve.

7. The needle assembly of claim 1 wherein said inner sleeve is made of a plastic material having a lower modulus than said plastic material of said outer sleeve.

8. The needle of claim 7 wherein said outer sleeve is made substantially of a polyolefin material.

9. The needle assembly of claim 7 wherein said hub assembly includes a hub and a post extending from the distal and proximal ends of said hub, wherein said post is made of a material capable of bonding with said inner sleeve material.

10. The needle assembly of claim 9 wherein said post includes a sealing ring and said bonding of said inner sleeve to said post provides a sterile barrier.

11. The needle assembly of claim 10 wherein prior to engagement of said cover with said hub assembly the inside diameter of said inner sleeve portion is smaller than the diameter of said sealing ring.

12. The needle assembly of claim 9 wherein said cover, initially removed from said assembly, is adapted for secure reattachment to said assembly.

13. The needle assembly of claim 12 wherein said secure reattachment of said cover to said post is evidenced by an audible clicking sound.

14. A needle cover for a medical needle, said cover having a proximal end and a distal end, said cover comprising:
an outer sleeve portion made of a first, plastic material and having an open proximal end;
an inner sleeve portion made of a second plastic material and associated with said outer sleeve portion having an open proximal end, substantially coterminous with said outer sleeve proximal end;
said outer sleeve portion having an inner diameter greater than the outer diameter of inner sleeve portion at said proximal ends.

15. The needle cover of claim 14 wherein the inner surface said outer sleeve portion includes an outwardly extending step at the proximal end of said inner surface.

16. The needle cover of claim 14 wherein said second plastic material has a modulus lower than the modulus of said first plastic material.

17. The needle cover of claim 14 wherein said first plastic material is a polyolefin, and said second plastic material comprises polyvinyl chloride.

18. The needle cover of claim 17 wherein said first plastic material comprises polypropylene.

19. The needle cover of claim 14 wherein said the outer surface of said outer sleeve portion includes a plurality of axially extending ribs.

20. The needle cover of claim 14 wherein said outer sleeve portion includes at least one window through which said inner sleeve portion is exposed to the outside environment.

21. The needle cover of claim 20 wherein said outer surface of said inner sleeve portion includes raised at least one projection disposed within said window.

22. A needle cover for a medical needle, said cover having a proximal end and a distal end, said cover comprising:
an outer sleeve portion made of a first plastic material and having an open proximal end;
an inner sleeve portion associated with said outer sleeve made from a second plastic material, having an open proximal end;
at least one window in said outer sleeve portion through which said inner sleeve is exposed.

23. The needle cover of claim 22 wherein said outer surface of said inner sleeve portion includes at least one projection disposed within said at least one window of said outer sleeve.

24. The needle cover of claim 23 wherein the outside diameter of inner sleeve portion projection is substantially flush with the part of said outer surface of said outer sleeve portion that is adjacent to said window.

25. A needle cover for a medical needle comprising: an elongated body having an outer surface and an inner surface, an open proximal end for receiving a needle and a closed distal end, said body defining an interior bore, wherein said inner surface has a modulus lower than the modulus of said outer surface.

26. The needle cover of claim 25 wherein the inner surface material has a Young's modulus of approximately 5,000-35,000 psi.

27. The needle cover of claim 26 wherein said body includes a bore for receiving a needle.

28. The needle cover of claim 26 wherein said outer surface includes windows to expose areas of said inner surfaces to the outside environment.

29. The needle cover of claim 25 wherein said outer surface includes at least one gripping member.

30. The needle cover of claim 29 wherein said outer surface includes a plurality of gripping members.

31. The needle cover of claim 30 wherein said outer surface includes a plurality of axially extending ribs.

32. The needle cover of claim 31 wherein said outer surface includes windows to expose areas of said inner surface to the environment.

33. The needle cover of claim 32 wherein said windows are disposed between said ribs.

34. A medical needle assembly comprising:
a needle cover;
a needle hub assembly including a hub and needle post extending through the interior of said hub and a medical needle attached to said post;
said post including a beveled sealing ring adapted for engaging said needle cover;
said hub including gripping members on the outer surface thereof.

35. The needle assembly of claim 34 wherein said hub includes sidewalls with said gripping members disposed thereon.

36. The needle assembly of claim 35 wherein said gripping members comprise a plurality of spaced apart raised ribs.

37. The needle assembly of claim 34 wherein said hub is generally cylindrical.

38. The needle assembly of claim 34 wherein said hub includes sidewalls that are generally concave relative to the longitudinal axis of said hub.
